(19) United States
(12) Patent Application Publication
(10) Pub. No.: US 2025/0041087 A1
(43) Pub. Date: Feb. 6, 2025

(54) STENT GRAFT

(71) Applicant: Japan Lifeline Co., Ltd., Tokyo (JP)

(72) Inventors: Yuki TOYODA, Tokyo (JP); Junya NAGANO, Tokyo (JP)

(21) Appl. No.: 18/739,972

(22) Filed: Jun. 11, 2024

(30) Foreign Application Priority Data
Aug. 2, 2023 (JP) 2023-126363

(51) Int. Cl. A61F 2/95; A61F 2/07

(52) U.S. Cl. CPC A61F 2/9522; A61F 2/07; A61F 2002/9511

(57) ABSTRACT

A stent graft of the present disclosure includes a graft having a cylindrical shape and an end stent connected to at least an axial end of the graft. The end stent includes a hooking portion hooked on a stent constraining mechanism of a delivery device so as to constrain expansion of the end stent, the hooking portion includes a part protruding from the axial end of the graft to an outer side in an axial direction, and a protrusion length of the part is configured to be shortened by expansion of the axial end of the graft together with the end stent. The variable range of the protrusion length of the hooking portion may include zero.

### CROSS-REFERENCE TO RELATED APPLICATIONS

This application claims the benefit of priority to Japanese Patent Application Number 2023-126363 filed on Aug. 2, 2023. The entire contents of the above-identified application are hereby incorporated by reference.

### TECHNICAL FIELD

The present disclosure relates to a stent graft.

### BACKGROUND

JP 2019-510579 A discloses a stent graft including a cylindrical graft and an end stent connected to at least the axial end of the graft. A delivery device as described in JP 2019-510579 A is used to transport the stent graft to a target position of a hollow organ. The delivery device includes a cover that constrains the expansion of the entire graft by covering the stent graft, and a stent constraining mechanism that constrains the expansion of the end stent by hooking the end stent.

### SUMMARY

To hook the end stent on the stent constraining mechanism, the hooking portion that is a part of the end stent needs to protrude from the axial end of the graft. When the end stent is expanded with the axial end of the graft due to the releasing of the constraint by the stent constraining mechanism, the protrusion length of the hooking portion of the end stent usually remains constant. Under such a structure, when the stent graft is stuck to the hollow organ by expanding the graft, defects caused by the protrusion of the end stent from the graft are likely to occur. The inventors of the present application have newly recognized such a problem, and have reached the concept of the stent graft of the present disclosure.

One of the objects of the present disclosure is to provide a stent graft capable of suppressing defects caused by protrusion of the end stent from the graft.

A stent graft of the present disclosure includes: a graft having a cylindrical shape; and an end stent connected to at least an axial end of the graft, wherein the end stent includes a hooking portion that is hooked on a stent constraining mechanism of a delivery device so as to constrain expansion of the end stent, and the hooking portion includes a part that protrudes from the axial end of the graft toward an outer side in an axial direction, and a protrusion length of the part can be shortened by expansion of the axial end together with the end stent.

According to the present disclosure, defects caused by the protrusion of the end stent from the graft can be suppressed.

### BRIEF DESCRIPTION OF DRAWINGS

FIG. 1A is a first schematic diagram for describing the effect of a stent graft of the present disclosure, and FIG. 1B is a second schematic diagram for the same.

FIG. 2A is a first schematic diagram for describing another effect of a stent graft of the present disclosure, and FIG. 2B is a second schematic diagram for the same.

FIG. 3 is a schematic perspective view of a delivery device of a first embodiment.

FIG. 4 is a schematic side sectional view around a stent graft in a delivery device of the first embodiment.

FIG. 5 is a first explanatory view of the operation of the delivery device of the first embodiment.

FIG. 6 is a second explanatory view of the operation of the delivery device of the first embodiment.

FIG. 7 is a side view schematically illustrating a stent graft of the first embodiment.

FIG. 8 is another side view schematically illustrating a stent graft of the first embodiment.

FIG. 9A is a development view schematically illustrating a part of a stent graft of the first embodiment, and FIG. 9B is a view in which a suture is omitted from FIG. 9A.

FIG. 10 is a view schematically illustrating one movable ridge portion of FIG. 9A together with a peripheral structure.

FIG. 11A is a diagram schematically illustrating the movable ridge portion of FIG. 10, and FIG. 11B is a diagram illustrating the operation of the movable ridge portion of FIG. 10.

FIG. 12 is a development view schematically illustrating a part of a stent graft of a second embodiment.

FIG. 13A is a development view schematically illustrating a part of a stent graft of a third embodiment. FIG. 13B is a view illustrating the operation of the part.

FIG. 14A is a development view schematically illustrating a part of the stent graft of the third embodiment. FIG. 14B is a view in which a suture is omitted from FIG. 14A.

### DESCRIPTION OF EMBODIMENTS

### First Embodiment

Embodiments for implementing a stent graft of the present disclosure will be described below. The same or equivalent elements will be denoted by the same reference numerals, and duplicate descriptions will be omitted. In each drawing, components will be omitted, enlarged, and reduced as appropriate for the convenience of description. Drawings are to be viewed in the orientation of the sign.

First, the stent graft of the present disclosure will be described from the background that led to the conception. In the delivery device, it is desired that the graft is constrained by the cover in a state where the graft contracts as uniformly as possible in a circumferential direction. This makes it advantageous to properly position the stent graft at a target position in the hollow organ and to smoothly expand the stent graft. To achieve this, it is desirable to contract the axial end of the graft with the end stent as uniformly as possible in a circumferential direction by hooking the end stent to the stent constraining mechanism of the delivery device. To hook the end stent on the stent constraining mechanism, a plurality of hooking portions of the end stent needs to protrude from the axial end of the graft.

As mentioned above, once the axial end of the graft expands from the contracted state due to the releasing of the constraint by the stent constraining mechanism, when the protrusion length of the hooking portion of the end stent remains constant, defects resulting from the protrusion of the end stent from the graft are likely to occur.

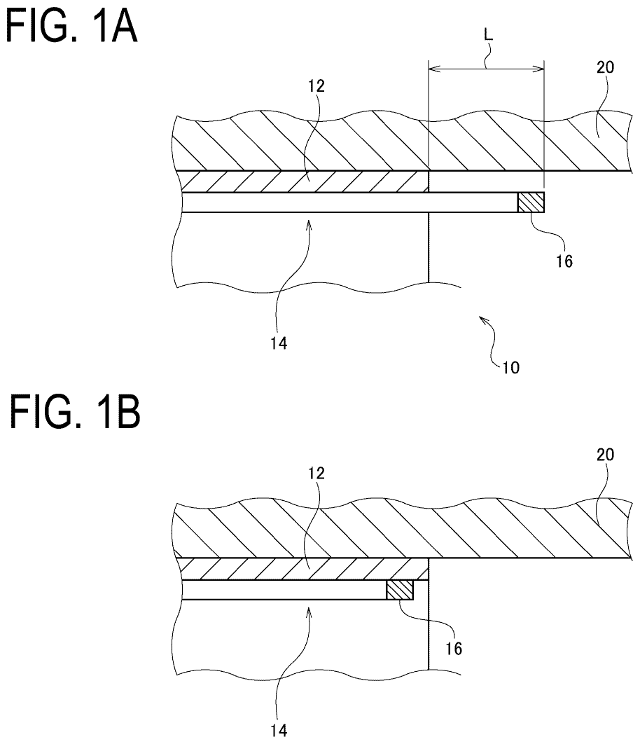

For example, a case will be considered where an end stent 14 is disposed inside a graft 12 as illustrated in FIG. 1A. In this case, when a stent graft 10 is stuck to a hollow organ 20, the contact area between the graft 12 and the end stent 14 decreases as a protrusion length L of a hooking portion 16 of the end stent 14 increases. As a result, defects that the expansion force of the protrusion part of the end stent 14 protruding from the graft 12 becomes difficult to be distributed by the graft 12 are likely to occur.

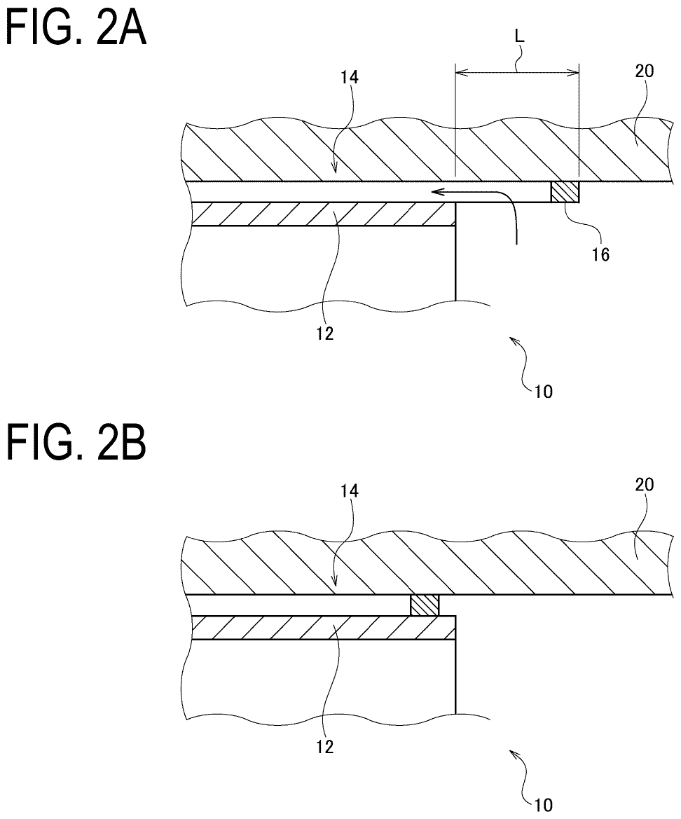

A case will be considered where the end stent 14 is disposed outside the graft 12 as illustrated in FIG. 2A. In this case, when the stent graft 10 is attached to the hollow organ 20, the longer the protrusion length L of the end stent 14, the easier it is for the body fluid to enter a wide area outside the graft 12 via the protrusion part of the end stent 14 protruding from the graft 12. As a result, defects that the adhesion of the graft 12 to the hollow organ 20 is reduced are likely to occur.

As a countermeasure, the hooking portion 16 of the stent graft according to the present disclosure is characterized in that the protrusion length L can be changed in conjunction with the expansion or contraction of the axial end of the graft 12 together with the end stent 14, and the protrusion length L can be shortened by the expansion of the axial end of the graft 12. Thus, due to the releasing of the constraint by the stent constraining mechanism, the protrusion length L of the hooking portion 16 can be shortened when an axial end 12a of the graft 12 expands. That is, the protrusion length L of the hooking portion 16 when the stent graft 10 is stuck to the hollow organ 20 can be shortened as compared with the protrusion length L of the hooking portion 16 when the hooking portion 16 is constrained by the stent constraining mechanism. That is, when the stent graft 10 is stuck into the hollow organ 20, the defects caused by the protrusion of the hooking portion 16 of the end stent 14 from the graft 12 can be suppressed, as compared with the case where the protrusion length L does not change before and after the constraint by the stent constraining mechanism is released.

For example, a case will be considered where the end stent 14 is arranged inside the graft 12 as illustrated in FIG. 1B. In this case, the shorter the protrusion length L of the hooking portion 16 of the end stent 14, the larger the contact area between the graft 12 and the end stent 14. This can suppress defects that it is difficult to distribute the expansion force of the hooking portion 16 of the end stent 14 to the graft 12. Thus, the easier it is to distribute the expansion force of the end stent 14 to the graft 12, the more advantageous it is that the burden on the hollow organ 20 can be reduced.

Additionally, a case will be considered where the end stent 14 is arranged outside the graft 12 as illustrated in FIG. 2B. In this case, the shorter the protrusion length L of the hooking portion 16 of the end stent 14 is, the more difficult it is for body fluid to enter through the inside of the protrusion part protruding from the graft 12. As a result, defects that the adhesion of the graft 12 to the hollow organ 20 is reduced can be suppressed.

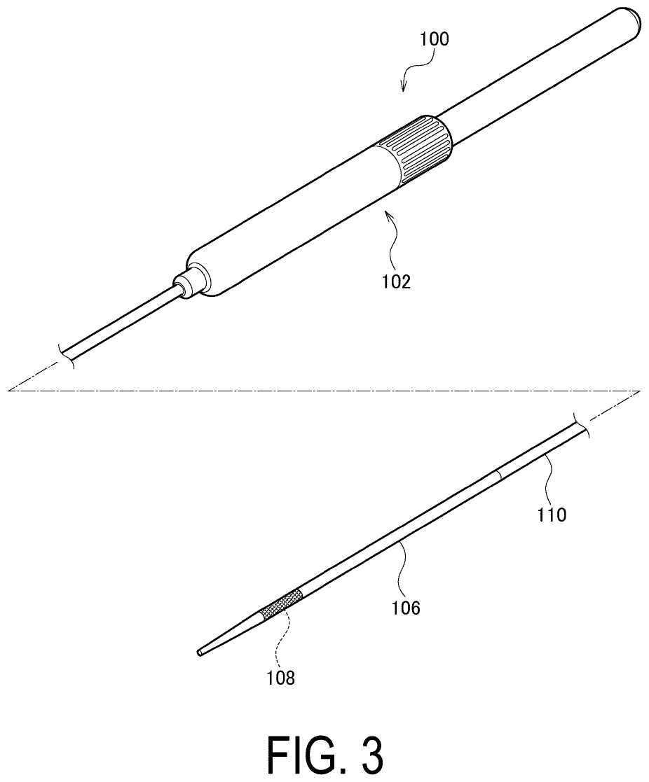

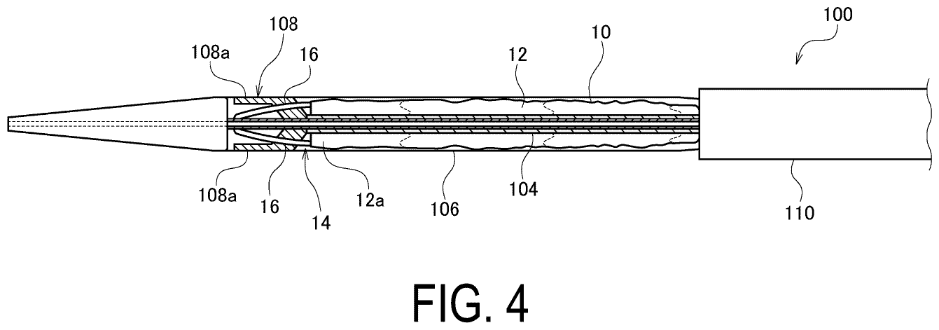

Description will be made with reference to FIGS. 3 and 4. Next, a delivery device 100 will be described. The stent graft 10 of FIG. 4 differs in appearance from the stent graft 10 of the embodiment for convenience of description.

The delivery device 100 includes a handle 102 gripped by an operator and a shaft 104 entering through the stent graft 10 and supporting the stent graft 10. Additionally, the delivery device 100 also includes a cover 106 that covers the stent graft 10 to constrain the expansion of the entire graft 12 and a stent constraining mechanism 108 that is hooked by a plurality of hooking portions 16 of the end stent 14 to constrain the expansion of the end stent 14. In addition, the delivery device 100 of the present embodiment includes an introducer sheath 110 connected to the handle 102.

The cover 106 enters through the introducer sheath 110 and can be pulled axially by operation of the operator using a first operation mechanism (not illustrated). The entire graft 12 is likely to expand with at least one stent including the end stent 14 of the stent graft 10. The cover 106 constrains the expansion of the entire graft 12 by the stent.

The stent constraining mechanism 108 is provided separately from the cover 106. The stent constraining mechanism 108 includes a plurality of stent receptacles 108a at which each of the plurality of hooking portions 16 of the end stent 14 is hooked. The plurality of stent receptacles 108a form a rod extending axially, and the hooking portions 16 of the end stent 14 are hooked through the inner side in the radial direction thereof. When the hooking portions 16 of the end stent 14 are hooked on the plurality of stent receptacles 108a, thereby constraining the expansion of the end stent 14, the expansion of the axial end 12a of the graft 12 is constrained as well. The plurality of stent receptacles 108a can be moved axially by the operation of an operator using a second operation mechanism (not illustrated). The stent constraining mechanism 108 can switch whether the end stent 14 is constrained by the axial movement of each stent receptacle 108a.

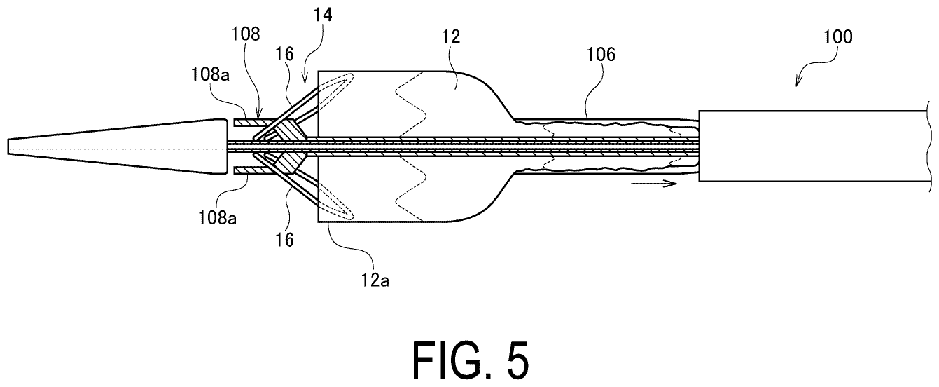

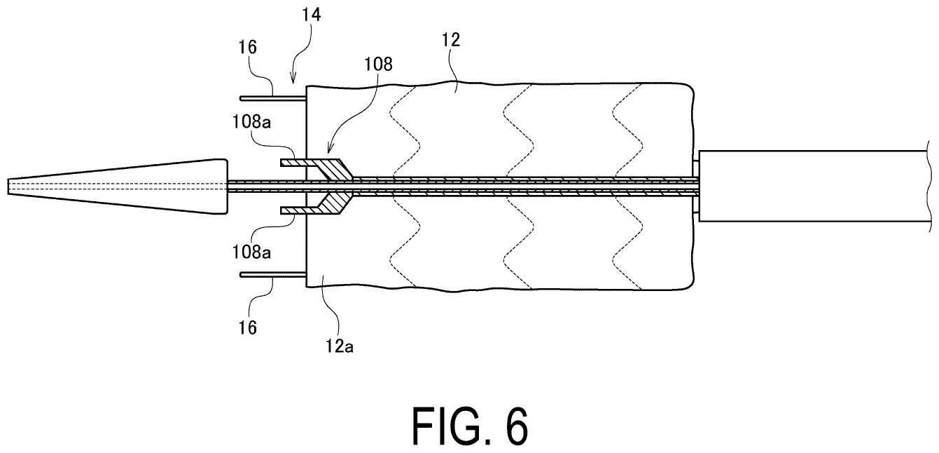

An example of a method of detaining the stent graft 10 by using the delivery device 100 will be described below. First, the stent graft 10 is conveyed together with the delivery device 100 to a target position of the hollow organ while the graft 12 is constrained in the contraction state by the delivery device 100. Next, as illustrated in FIG. 5, the constraint of the graft 12 by the cover 106 is released by pulling the cover 106 with the first operation mechanism. Thereafter, as illustrated in FIG. 6, each stent receptacle 108a of the stent constraining mechanism 108 is moved axially by the second operation mechanism to release the hooking of the end stent 14 by the stent constraining mechanism 108 and to release the constraint of the end stent 14 by the stent constraining mechanism 108. As a result, the stent graft 10 is stuck at the target position of the hollow organ by expanding the graft 12 from the contracted state.

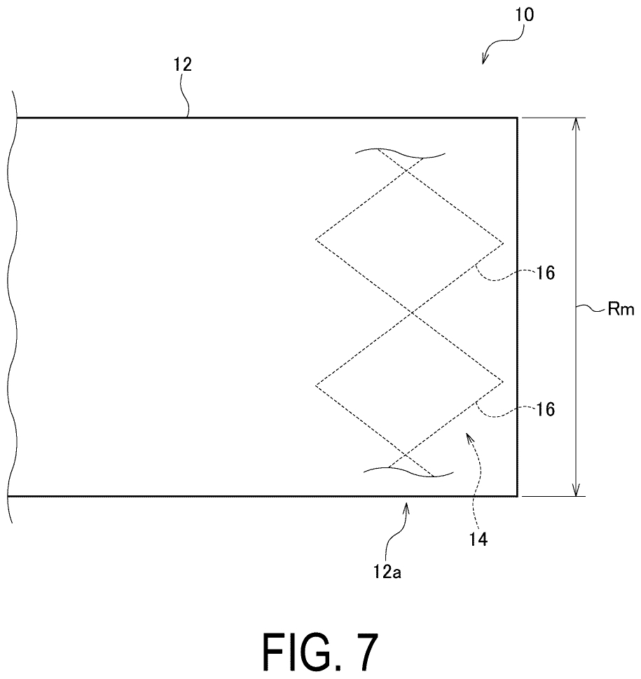

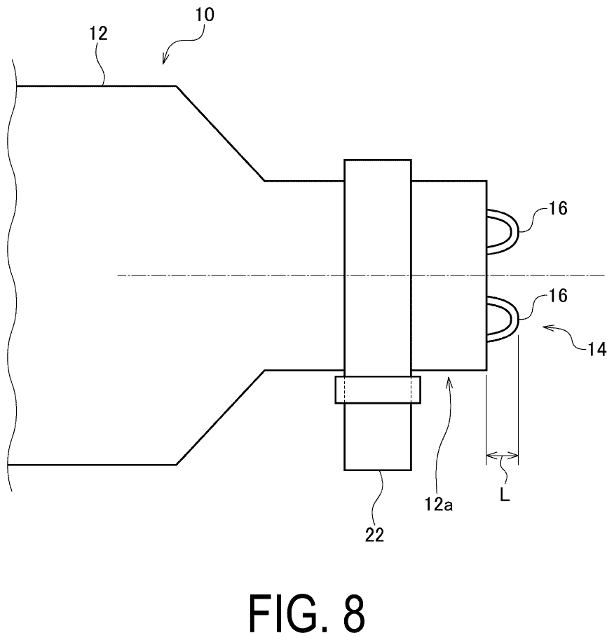

Description will be made with reference to FIGS. 7 and 8. Next, the stent graft 10 will be described. FIG. 7 schematically illustrates a state in which the graft 12 of the stent graft 10 is in a maximum expansion state (described below), and FIG. 8 schematically illustrates a state in which the graft 12 is in a reference contraction state (described below). In the present specification, a direction along the center line of the graft 12 is called an axial direction, and a radial direction and a circumferential direction with the center line as a circular center are called simply a radial direction and a circumferential direction. In the present specification, the axial direction is sometimes called the “outer side” or “inner side” in the axial direction of the graft 12. Here, the “outer side in the axial direction” refers to the side away from the center position in the axial direction of the graft 12, and the “inner side in the axial direction” refers to the side approaching the center position in the axial direction of the graft 12.

The stent graft 10 is placed in the hollow organ of the body for the treatment of vascular dissection, aneurysm, obstruction of the digestive tract, and the like. The hollow organ here includes, for example, a blood vessel and the digestive tract. The stent graft 10 includes a cylindrical graft 12 and the end stent 14 connected to at least the axial end 12a of the graft 12. The stent graft 10 may include other stents connected to the inner side in the axial direction relative to the end stent 14. In addition, the end stent 14 may extend to the center portion in the axial direction of the graft 12. The end stent 14 of the present embodiment is disposed inside the graft 12, but may also be disposed outside.

The graft 12 is a flexible cylindrical member. The graft 12 expands with the end stent 14 to maintain its stuck state within the hollow organ 20, thereby forming a flow channel through which body fluid flows. The graft 12 is composed of, for example, a knitted fabric or a woven fabric using resin fibers, metal fibers, or the like.

The end stent 14 is expandable outside in the radial direction with the graft 12 by self-expansion. Here, self-expansion means expansion due to the restoring force associated with its own elastic deformation. The end stent 14 is composed of a metal such as a shape memory alloy, stainless steel, and a wire material using resin or the like. Hereinafter, a state in which no external force for contraction is applied to the graft 12 and the end stent 14, and the axial end 12a of the graft 12 and the end stent 14 expand until the outer diameter of the graft 12 is maximum due to the self-expansion force of the end stent 14, is called a maximum expansion state.

The end stent 14 is provided with a plurality of hooking portions 16 which are hooked on the stent constraining mechanism 108 of the delivery device 100 to constrain the expansion of the end stent 14. The hooking portions 16 of the present embodiment protrude to the outer side in the axial direction of the graft 12 and are folded back in the axial direction. The plurality of hooking portions 16 are arranged in the circumferential direction at the end stent 14. Only two hooking portions 16 are illustrated schematically here, but the number is not particularly limited and may be either singular or three or more.

When the axial end 12a of the graft 12 expands or contracts with the end stent 14, the plurality of hooking portions 16 can change the protrusion length L of the part protruding from the axial end 12a of the graft 12 toward the outer side in the axial direction in conjunction with the expansion or contraction. The configuration for achieving this will be described below. The protrusion length L here refers to the length along the axial direction from an outer end in the axial direction of the hooking portion 16 of the end stent 14 to the end in the axial direction of the graft 12 at the same position in the circumferential direction as the outer end in the axial direction. FIG. 7 illustrates an example in which the protrusion length L is zero. When the hooking portion 16 protrudes from the axial end 12a of the graft 12, the protrusion location of the hooking portion 16 can hook to the stent receptacle 108a of the stent constraining mechanism 108. At this time, an opening that penetrates the graft 12 in the radial direction is formed between the protrusion of the hooking portion 16 and the axial end 12a of the graft 12. The stent receptacle 108a of the stent constraining mechanism 108 passes through the opening formed by the hooking portion 16, thereby hooking the hooking portion 16 to the stent receptacle 108a.

The plurality of hooking portions 16 can shorten the protrusion length L by the expansion of the axial end 12a of the graft 12. The hooking portion 16 can gradually shorten the protrusion length L until the lower limit value of the variable range of the protrusion length L is reached by the expansion of the axial end 12a of the graft 12. The variable range of the protrusion length L includes zero. It can be said that the variable range of the protrusion length L makes zero the lower limit value. The hooking portion 16 can be gradually retracted from the axial end of the end stent 14 to the inner side in the axial direction by the expansion of the axial end 12a until the graft 12 reaches the maximum expansion state after the protrusion length L reaches zero. In addition, the protrusion length L of the plurality of hooking portions 16 may be zero when the maximum expansion state is reached.

The above effects of the stent graft 10 will be described. As described above, the hooking portion 16 of the stent graft 10 can shorten the protrusion length L by the expansion of the graft 12 together with the end stent 14. Thus, as described above, when the stent graft 10 is stuck to the hollow organ 20, the protrusion length L of the hooking portion 16 can be shortened to suppress the defects caused by the protrusion of the end stent 14. Furthermore, when the end stent 14 is attached to the stent constraining mechanism 108, the attachability of the end stent 14 to the stent constraining mechanism 108 can be improved by increasing the protrusion length L of the hooking portion 16.

The protrusion length L of the end stent 14 includes zero in the variable range. Thus, as compared with the case where the variable range of the protrusion length L is greater than zero, the protrusion length L of the end stent 14 when the stent graft 10 is stuck to the hollow organ 20 can be shortened as much as possible. Consequently, the suppression effect of the defects caused by the protrusion of the end stent 14 can be acquired favorably.

Next, other features of the stent graft 10 will be described. A maximum protrusion length and an average protrusion length of the hooking portion 16 will be considered. When the end stent 14 has only a single hooking portion 16, the protrusion length L of the single hooking portion 16 is to be the maximum protrusion length and the average protrusion length. When the end stent 14 has a plurality of hooking portions 16, the maximum of the protrusion lengths L of the plurality of hooking portions 16 is to be the maximum protrusion length, and the simple average of the protrusion lengths is to be the average protrusion length. At this time, the stent graft 10 can shorten each of the maximum protrusion length and the average protrusion length of the hooking portions 16 by the expansion of the graft 12, and the variable range may include zero.

An outer diameter of the axial end 12a of the graft 12 when the graft 12 is in the maximum expansion state is called Rm. The outer diameter in the present specification refers to the diameter. The state in which the outer diameter of the axial end 12a of the graft 12 contracts to 50% of the outer diameter Rm in the maximum expansion state is called a reference contraction state. The state in which the outer diameter of the axial end 12a of the graft 12 is Rm×50% is to be the reference contraction state. In this reference contraction state, the contraction part of the axial end 12a of the graft 12 extends straight along the axial direction.

A maximum protrusion length L1 of the hooking portion 16 when the graft 12 is in the reference contraction state is preferably no less than 1 mm. By satisfying this condition, when the end stent 14 is attached to the stent constraining mechanism 108, the protrusion length of the hooking portion 16 can be properly increased, and the attachability of the end stent 14 to the stent constraining mechanism 108 can be improved. The upper limit value of the maximum protrusion length L1 of the hooking portion 16 is not particularly limited but may be, for example, 10 mm.

A maximum protrusion length L0 of the hooking portion 16 when the graft 12 is in the maximum expansion state is preferably no more than 5 mm. Thus, when the stent graft 10 is stuck to the hollow organ 20, the protrusion length of the hooking portion 16 can be properly shortened, and the suppression effect of the defects caused by the protrusion of the end stent 14 can be favorably acquired.

When the end stent 14 is shortened from the maximum protrusion length L1 in the reference contraction state to the maximum protrusion length L0 in the maximum expansion state, the ratio of the shortening degree of the maximum protrusion length to the maximum protrusion length L1 is called a shortening ratio (%). The shortening ratio is expressed from the following equation (1) and varies within the range from greater than zero to no more than 100. When the maximum protrusion length L0 of the end stent 14 in the maximum expansion state is zero, the shortening ratio is the maximum value (100%).

Shortening ratio (%)={(L1−L0)/L1}×100 (1)

The shortening ratio is preferably no less than 50%. Thus, when the end stent 14 is attached to the stent constraining mechanism 108, the protrusion length of the end stent 14 can be relatively properly increased, while the stent graft 10 is stuck to the hollow organ 20, the protrusion length of the end stent 14 can be relatively properly decreased. Thus, while the attachability of the end stent 14 to the stent constraining mechanism 108 is improved, the suppression effect of the defects caused by the protrusion of the end stent 14 can be favorably acquired.

The measurement of the parameters (outer diameter of graft 12, protrusion length of end stent 14) related to the stent graft 10 will be described below. In measuring the parameters, to bring the graft 12 into the reference contraction state, a binding band 22 may be wound around the axial end 12a of the graft 12, and the graft 12 may be contracted evenly in the whole circumferential range by the binding band 22. The outer diameter of the axial end 12a of the graft 12 may adopt the maximum value of the measured value when measured over the whole circumference by a non-contact outer diameter measuring instrument. When the graft 12 is brought into the reference contraction state by the binding band 22, the binding band 22 may be composed of a translucent material, and the outer diameter at the position where the binding band 22 is wound may be measured by an outer diameter measuring instrument using a laser beam.

In evaluating the parameters (maximum protrusion length L1, shortening ratio) when the graft 12 is in the reference contraction state described so far, each of the lower and upper limit values as a reference is called the reference lower limit value and the reference upper limit value. In terms of the maximum protrusion length L1, 1 mm is the reference lower limit value and 10 mm is the reference upper limit value. In terms of the shortening ratio, 50% is the reference lower limit value. In determining the success or failure of the condition that the parameter in the reference contraction state is no less than the reference upper limit value, or no more than the reference lower limit value, the reference contraction state in which the outer diameter of the graft 12 is equal to Rm×50% may actually be achieved. In this case, for example, when the measured value of the maximum protrusion length L1 in the reference contraction state is no less than the reference lower limit value (1 mm), the condition that the maximum protrusion length L1 is no less than the reference lower limit value is determined to be satisfied. In addition, in consideration of the convenience of measurement, the outer diameter of the graft 12 may be close to Rm×50% as follows, and then the success or failure of the condition relating to the parameter may be determined.

The smaller the outer diameter of the axial end 12a of the graft 12, the longer the maximum protrusion length L1 of the hooking portion 16 and the greater the shortening ratio. The larger the outer diameter, the shorter the maximum protrusion length L1 of the hooking portion 16 and the smaller the shortening ratio. Here, a case will be considered where the outer diameter of the graft 12 is within the range from greater than 50% to no more than 60% of the outer diameter Rm and is as close as possible to 50% of the outer diameter Rm. In this case, when the measured value of the maximum protrusion length L1 is no less than the reference lower limit value (1 mm in this case), the maximum protrusion length L1 in the reference contraction state, which is equal to 50% relative to the outer diameter Rm, is also no less than the reference lower limit value (because L1 becomes longer as the outer diameter of the graft 12 becomes smaller). Similarly, in this case, when the measured value of the shortening ratio is no less than the reference lower limit value (50% in this case), the shortening ratio in the reference contraction state, which is equal to 50% relative to the outer diameter Rm, is also no less than the reference lower limit value. Based on this, when the outer diameter of the graft 12 is within a range from greater than 50% to no more than 60% of the outer diameter Rm and as close as possible to 50% of the outer diameter Rm, once the measured value of the maximum protrusion length L1 or the shortening ratio is no less than the reference lower limit value, it may be determined that the maximum protrusion length L1 or the shortening ratio in the reference contraction state is also no less than the reference lower limit value.

Furthermore, a case will be considered where the outer diameter of the graft 12 is within a range from no less than 40% to less than 50% of the outer diameter Rm and is as close as possible to 50% of the outer diameter Rm. In this case, when the measured value of the maximum protrusion length L1 of the hooking portion 16 is no more than the reference upper limit value (10 mm in this case), the maximum protrusion length L1 in the reference contraction state, which is equal to 50% relative to the outer diameter, is also no more than the reference upper limit value (because L1 becomes shorter as the outer diameter of the graft 12 becomes larger). Based on this, when the outer diameter of the graft 12 is within a range from no less than 40% to less than 50% of the outer diameter Rm and as close as possible to 50% of the outer diameter Rm, once the measured value of the maximum protrusion length L1 is no more than the reference upper limit value, it may be determined that the maximum protrusion length L1 in the reference contraction state is also no more than the reference upper limit value.

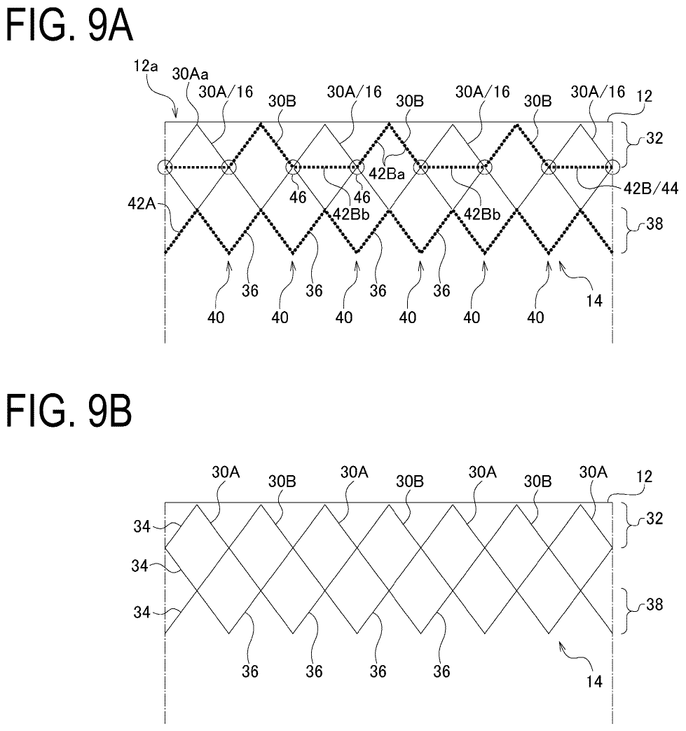

Description will be made with reference to FIGS. 9A and 9B. FIG. 9A is a development view illustrating a part of a shape in which a part of the stent graft 10 is cut open along an axial direction and expanded in a plane. The dashed lines in FIG. 9A indicate sutures 42A and 42B.

The end stent 14 is provided with a plurality of ridge portions 30A and 30B which protrude to the outer side in the axial direction of the graft 12 and are arranged in the circumferential direction. In the present embodiment, all the ridge portions 30A and 30B form an outer zigzag pattern 32 which extends in a zigzag shape in the axial direction in the circumferential direction by the adjacent ridge portions 30A and 30B contacting each other. The outer zigzag pattern 32 of the present embodiment is annularly continuous. The ridge portions 30A and 30B adjacent to each other in the circumferential direction may be separated from each other.

The plurality of ridge portions 30A and 30B are formed by at least one wave portion 34 that extends in the circumferential direction and forms a wave shape while meandering into the axial direction. The plurality of ridge portions 30A and 30B of the present embodiment are formed by a plurality (three in detail) of wave portions 34. The plurality of wave portions 34 of the present embodiment form wave shapes of the same wavelength and different phases from each other. In the present embodiment, the plurality of wave portions 34 are formed of one wire rod that repeatedly circles the graft 12 but may be formed of separate wire rods. The wave formed by the wave portions 34 is not particularly limited as long as forming a shape that extends in the circumferential direction while meandering in the axial direction. Although an example in which the wave portions 34 form a triangular wave is illustrated here, various wave shapes (e.g., rectangular wave shape, or the like) adopted in the end stent 14 may be formed.

The plurality of ridge portions 30A and 30B include at least one movable ridge portion 30A and at least one fixed ridge portion 30B. In the present embodiment, the movable ridge portions 30A and the fixed ridge portions 30B are alternately arranged toward the circumferential direction, but the arrangement order is not particularly limited. The movable ridge portion 30A is movable relative to the graft 12 in at least an apex portion 30Aa in the axial direction as described below, and constitutes the hooking portion 16 for being hooked on the stent constraining mechanism 108 described above. The fixed ridge portion 30B is fixed to the graft 12 relatively immovably in the axial direction and the circumferential direction by being connected by a connecting member 44 described below. The relative movement here refers to the mentioned two portions (e.g., the graft 12 and the apex portion 30Aa of the movable ridge portion 30A) moving relative to each other.

The end stent 14 of the present embodiment includes a plurality of valley portions 36 which protrude to the inner side in the axial direction of the graft 12 and are arranged in the circumferential direction. The plurality of valley portions 36 are formed by at least one (here three) wave portion 34 described above. In the present embodiment, all valley portions 36 form an inner zigzag pattern 38 which extends in a zigzag shape in the axial direction toward the circumferential direction by the adjacent valley portions 36 contacting each other. The inner zigzag pattern 38 of the present embodiment is annularly continuous.

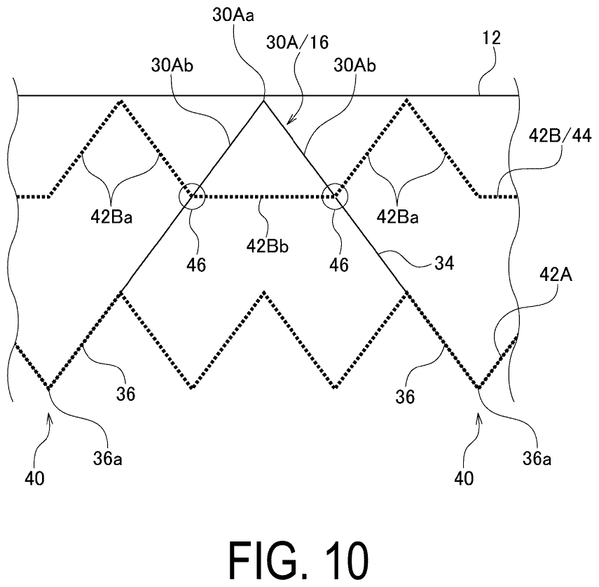

Description will be made with reference to FIGS. 9A, 9B and 10. The end stent 14 includes a plurality of fixed portions 40, which are continuously provided at the movable ridge portion 30A on both sides of the circumferential direction relative to the movable ridge portion 30A and are fixed to the graft 12. The fixed portion 40 of the present embodiment is provided at a part including an apex portion 36a in the valley portion 36 of the wave portion 34. Each movable ridge portion 30A has a pair of fixed portions 40 corresponding to one movable ridge portion 30A. In the present embodiment, the fixed portion 40 corresponding to one movable ridge portion 30A is not common to the fixed portion 40 corresponding to another movable ridge portion 30A but may be common.

The fixed portion 40 is connected to the graft 12 by being sutured, bonded, or the like, so as to be fixed to the graft 12 relatively immovably in the circumferential direction and the axial direction. The fixed portion 40 of the present embodiment is connected to the graft 12 by suturing using a first suture 42A. The first suture 42A, in the present embodiment, is used for suturing a part of the end stent 14 to the graft 12 along the inner zigzag pattern 38 described above and fixing a plurality of fixed portions 40 on the inner zigzag pattern 38 to the graft 12.

The movable ridge portion 30A includes the apex portion 30Aa constituting the outer end in the axial direction of the movable ridge portion 30A, and a pair of intermediate line portions 30Ab provided between the apex portion 30Aa and the fixed portions 40. The movable ridge portion 30A is movable relative to the graft 12 in the axial direction in at least the apex portion 30Aa. To achieve this, the stent graft 10 of the present embodiment includes the connecting member 44, which connects the intermediate line portion 30Ab to the graft 12 so as to move in a length direction of the intermediate line portion 30Ab relative to the graft 12 located on the inner side in the axial direction relative to the apex portion 30Aa of the movable ridge portion 30A. The length direction here refers to the extending direction (axial direction) of the intermediate line portion 30Ab. In addition, to achieve this, the movable ridge portion 30A may not be connected to the graft 12 by suturing, bonding, or the like.

The connecting member 44 of the present embodiment connects, to the graft 12, each of the pair of intermediate line portions 30Ab on both sides of the apex portion 30Aa of the movable ridge portion 30A in the circumferential direction. Alternatively, only one of the intermediate line portions 30Ab may be connected to the graft 12. The connecting member 44 of the present embodiment is a second suture 42B. The second suture 42B is used for sewing the graft 12 to form an eye 46 of one thread that is used for suturing the intermediate line portion 30Ab to the graft 12. The position of the eye 46 of one thread is given with a circle. The intermediate line portion 30Ab is connected relatively movably in the length direction relative to the graft 12 by being sutured in one position (eye 46 of one thread) with the second suture 42B.

The second suture 42B is used for sewing the graft 12, thereby forming a first seam portion 42Ba extending along the fixed ridge portion 30B and a second seam portion 42Bb extending between the pair of intermediate line portions 30Ab of the movable ridge portion 30A. In each of the seam portions 42Ba and 42Bb, a plurality of thread eyes are repeatedly arranged in a direction along the seam portions 42Ba and 42Bb. The second suture 42B connects, to the graft 12, the plurality of fixed ridge portions 30B on the outer zigzag pattern 32 with the first seam portion 42Ba. The second suture 42B connects, to the graft 12, the intermediate line portions 30Ab on the outer zigzag pattern 32 by the aforementioned thread eyes 46 provided at the end of the second seam portion 42Bb.

The fixed ridge portion 30B is connected to the graft 12 by being sutured, bonded, or the like, thereby being fixed to the graft 12 in the axial direction and the circumferential direction relatively immovably. The fixed ridge portion 30B of the present embodiment is connected to the graft 12 by suturing with the first seam portion 42Ba of the second suture 42B described above. The fixed ridge portion 30B of the present embodiment is connected to the graft 12 in the axial direction range including the apex portion. The fixed ridge portion 30B of the present embodiment is connected to the graft 12 on the outer side in the axial direction relative to the connection position (position of the thread eye 46) of the movable ridge portion 30A by the connecting member 44.

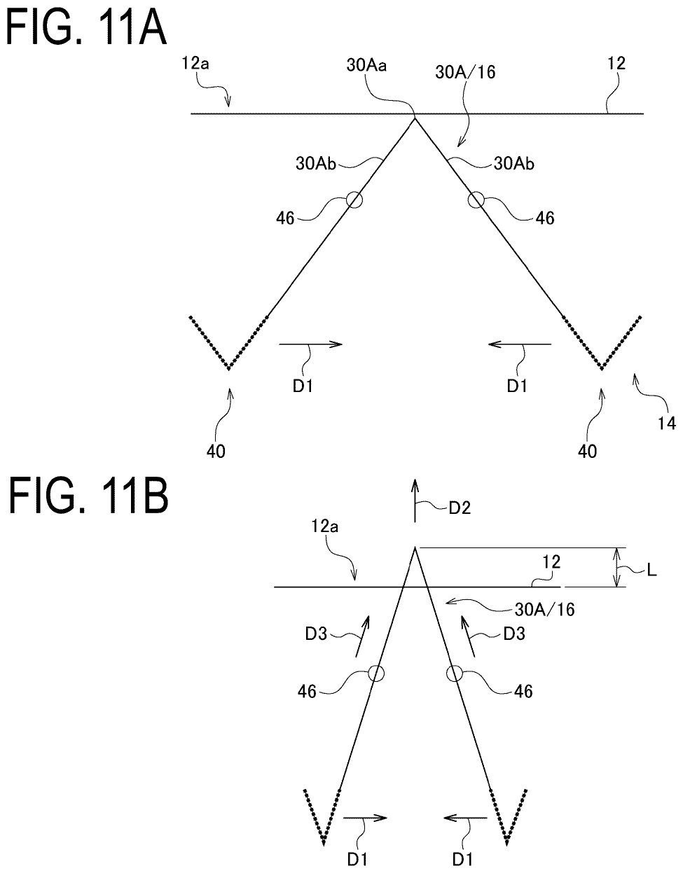

The above operational effect of the stent graft 10 will be described. Description will be made with reference to FIGS. 11A and 11B. As described above, the end stent 14 includes a pair of fixed portions 40, which are continuously provided at the movable ridge portion 30A on both sides of the circumferential direction relative to the movable ridge portion 30A and are fixed to the graft 12. Thus, when the graft 12 contracts, the pair of fixed portions 40 corresponding to the movable ridge portion 30A of the end stent 14 also moves in a direction D1 together with the graft 12 so that the distance in the circumferential direction between the pair of fixed portions 40 is shortened. Thus, as the distance in the circumferential direction between the pair of fixed portions 40 becomes shorter, the apex portion 30Aa of the movable ridge portion 30A between the pair of fixed portions 40 relatively moves in a direction D2 toward the outer side in the axial direction of the graft 12. Accordingly, the protrusion length L of the movable ridge portion 30A protruding from the axial end of the graft 12 toward the outer side in the axial direction can be increased.

When the graft 12 expands, the pair of fixed portions 40 of the end stent 14 also move together with the graft 12 so that the distance in the circumferential direction between the pair of fixed portions 40 is increased. Thus, as the distance in the circumferential direction between the pair of fixed portions 40 increases, the apex portion 30Aa of the movable ridge portion 30A relatively moves toward the inner side in the axial direction of the graft 12. Accordingly, the protrusion length L of the movable ridge portion 30A protruding from the axial end of the graft 12 toward the outer side in the axial direction can be shortened.

That is, by moving the apex portion 30Aa of the movable ridge portion 30A relatively in the axial direction in conjunction with the expansion and contraction of the graft 12, the protrusion length L of the movable ridge portion 30A relative to the axial end of the graft 12 can be changed. In other words, by adopting such a configuration, the movable ridge portion 30A is configured as the hooking portion 16 capable of changing the protrusion length relative to the axial end 12a of the graft 12.

The stent graft 10 includes the connecting member 44 that connects the intermediate line portion 30Ab to the graft 12 relatively movably in the length direction of the intermediate line portion 30Ab of the movable ridge portion 30A relative to the graft 12. Thus, when the apex portion 30Aa of the movable ridge portion 30A relative to the graft 12 is about to move in the axial direction, the relative position of the movable ridge portion 30A relative to the graft 12 can be held at the connection position (position marked with a circle in the illustration) by the connecting member 44 while allowing movement of the intermediate line portion 30Ab relative to the graft 12 in the length direction. For example, a case will be considered where a pair of fixed portions 40 corresponding to the movable ridge portion 30A move in the direction D1. In this case, the intermediate line portion 30Ab can be moved in a direction D3 along the length direction relative to the graft 12 at the connection position by the connecting member 44, and the situation where the whole movable ridge portion 30A is greatly inclined relative to the axial direction can be avoided. Thus, the apex portion 30Aa of the movable ridge portion 30A can be moved relative to the graft 12 along the axial direction as much as possible, and the protrusion length L of the movable ridge portion 30A can be stably changed in conjunction with the expansion and contraction of the graft 12.

The plurality of ridge portions 30A and 30B include the fixed ridge portion 30B fixed to the graft 12, in addition to the movable ridge portion 30A capable of changing the protrusion length relative to the graft 12. Thus, the range in which the axial end 12a of the graft 12 is connected to the end stent 14 can be widened as compared with the case where the plurality of ridge portions are only the movable ridge portions 30A. In particular, by connecting the fixed ridge portion 30B onto the outer side in the axial direction relative to the connection position of the movable ridge portion 30A, the range in the axial direction in which the axial end 12a of the graft 12 is connected to the end stent 14 can be widened outside the axial direction, as compared with the case where the fixed ridge portion 30B is not present. Consequently, the turning up of the axial end 12a of the graft 12 at the end can be suppressed.

To adjust the variable range of the protrusion length L of the movable ridge portion 30A, (1) the distance in the circumferential direction of the pair of fixed portions 40 that are continuous with the movable ridge portion 30A, and (2) the angle of a line segment connecting the apex portion 30Aa of the movable ridge portion 30A and the fixed portion 40 relative to the axial direction may be adjusted. For example, as the distance in the circumferential direction of (1) is increased or as the angle of (2) is increased, the variable range of the protrusion length L of the movable ridge portion 30A can be increased.

### Second Embodiment

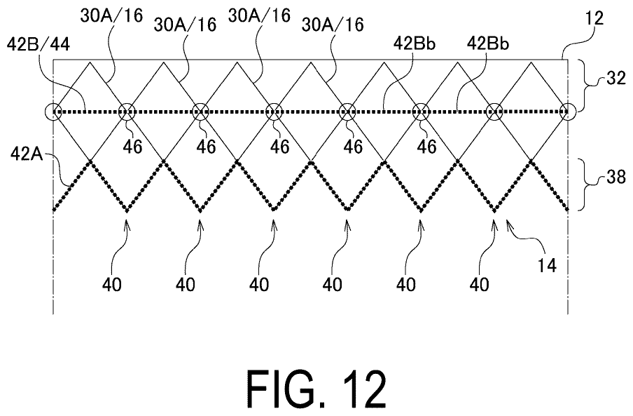

Description will be made with reference to FIG. 12. In the following embodiments, the same contents as in the first embodiment apply to the components that have been described in the first embodiment but are not described below.

In the above embodiments, the example has been described in which the movable ridge portion 30A and the fixed ridge portion 30B are alternately arranged in the plurality of ridge portions of the end stent 14. All of the plurality of ridge portions in the present embodiment are movable ridge portions 30A. Thus, the plurality of ridge portions may not include the fixed ridge portions 30B. Similar to the first embodiment, the plurality of movable ridge portions 30A of the present embodiment also have each of the pair of intermediate line portions 30Ab connected to the graft 12 by the connecting member 44 composed of the second suture 42B. Unlike the first embodiment, the second suture 42B is used for sewing the graft 12 so as to form only the aforementioned second seam portion 42Bb. In the present embodiment, the same effect as in the first embodiment can be acquired except for the features related to the fixed ridge portion 30B.

### Third Embodiment

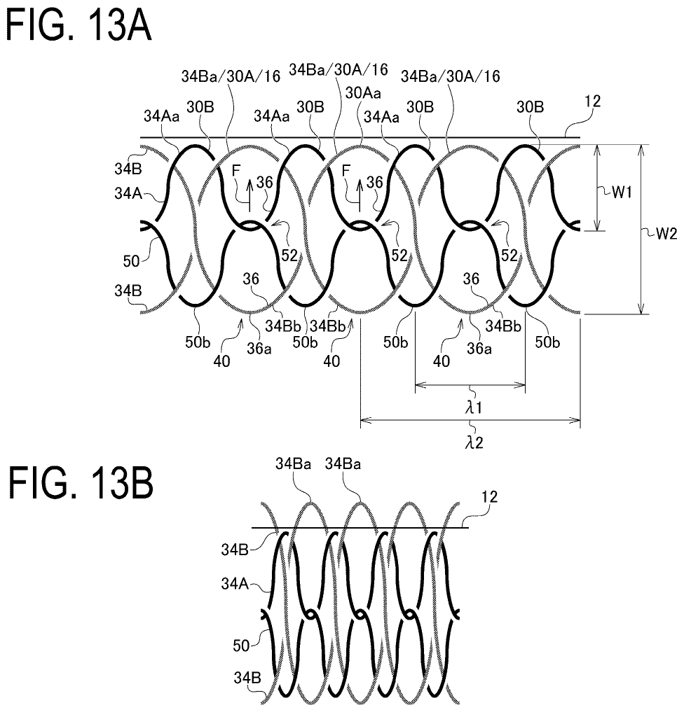

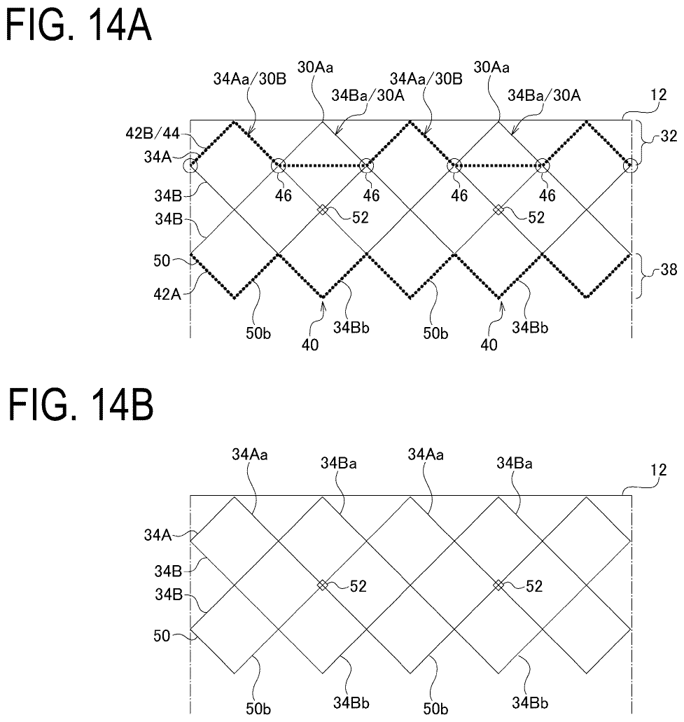

Description will be made with reference to FIGS. 13A, 13B, 14A, and 14B. FIG. 13A is a diagram in which the sutures 42A and 42B are omitted from FIG. 14A.

The plurality of ridge portions 30A and 30B in the present embodiment also include the movable ridge portions 30A and the fixed ridge portions 30B, as in the first embodiment, and the movable ridge portions 30A and the fixed ridge portions 30B are alternately arranged toward the circumferential direction. In the present embodiment, the plurality of wave portions 34A and 34B forming the ridge portions 30A and 30B include a first wave portion 34A and a second wave portion 34B forming a wave of a second wavelength λ2 longer than the first wavelength λ1 of the wave formed by the first wave portion 34A, respectively. In the present embodiment, there are two second wave portions 34B having the same wavelength and differing in phase by ½ of the wavelength. The two second wave portions 34B are illustrated to be formed of one wire rod that repeatedly orbits graft 12, but may be formed of separate wire rods.

The end stent 14 includes, in addition to a plurality of wave portions 34A and 34B forming ridge portions 30A and 30B, an inner wave portion 50 forming a wave shape having the same wavelength as the first wave portion 34A. The inner wave portion 50 differs in phase by ½ of the wavelength of the first wave portion 34A. The valley portion of the first wave portion 34A and the ridge portion of the inner wave portion 50 form a hook portion 52 that hooks to each other. In FIGS. 14A and 14B, the position of the hook portion 52 is indicated by a square symbol.

A ridge portion 34Aa of a first wave portion 34A is arranged between the ridge portions 34Ba of the second wave portion 34B adjacent in the circumferential direction and is in contact with ridge portions 34Ba. As a result, the ridge portions 34Aa of the first wave portion 34A and the ridge portions 34Ba of the second wave portion 34B form the outer zigzag pattern 32 extending in a zigzag shape in the circumferential direction.

A valley portion 50b of the inner wave portion 50 is arranged between the valley portions 34Bb of the second wave portion 34B adjacent in the circumferential direction and is in contact with a valley portion 34Bb. Thus, the valley portion 34Bb of the second wave portion 34B of the end stent 14 and the valley portion 50b of the inner wave portion 50 form the inner zigzag pattern 38 that extends in a zigzag shape in the circumferential direction.

To satisfy the aforementioned condition, (first wavelength λ1 of first wave portion 34A)<(second wavelength λ2 of second wave portion 34B), the minimum wavelength of each wave included in the second wave portion 34B may be longer than the maximum wavelength of each wave included in the first wave portion 34A. The second wavelength λ2 of the present embodiment is an integer multiple of the first wavelength λ1 (two times, as an example here), but may be any other multiple. To satisfy this condition, when a simple average of the wavelength of each wave included in the first wave portion 34A is defined as a first average wavelength and a simple average of the wavelength of each wave included in the second wave portion 34B is defined as a second average wavelength, the second average wavelength is sufficient in the range of +10% relative to an integer multiple of the first average wavelength. To satisfy the condition, (first wavelength λ1)<(second wavelength λ2), the specific long and short relationship is not particularly limited to the content of the embodiment.

The second wave portion 34B of the present embodiment forms a wave shape of a second amplitude W2 larger than a first amplitude W1 of the wave formed by the first wave portion 34A. To satisfy this condition, the minimum amplitude of each wave included in the second wave portion 34B may be larger than the maximum amplitude of each wave included in the first wave portion 34A. The second amplitude W2 of the present embodiment is an integer multiple of the first amplitude W1 (two times, as an example here), but may be any other multiple. To satisfy this condition, when an average value of the amplitude of each wave included in the first wave portion 34A is defined as a first average amplitude and an average value of the amplitude of each wave included in the second wave portion 34B is defined as a second average amplitude, the second average amplitude is sufficient in the range of +10% relative to an integer multiple of the first average amplitude. In satisfying the first wavelength λ1<the second wavelength λ2, there is no specific relationship between the first amplitude W1 and the second amplitude W2.

The movable ridge portion 30A of the present embodiment is constituted by the ridge portion 34Ba of the second wave portion 34B, and the fixed ridge portion 30B is constituted by the ridge portion 34Aa of the first wave portion 34A. A plurality of fixed portions 40 continuous to the movable ridge portion 30A of the end stent 14 are provided in the valley portion 34Bb of the second wave portion 34B. To achieve this, as described above, by suturing a portion of the end stent 14 to the graft 12 along the inner zigzag pattern 38 with the first suture 42A, a plurality of fixed portions 40 on the inner zigzag pattern 38 are fixed to the graft 12. At this time, the valley portion 50b of the inner wave portion 50 on the inner zigzag pattern 38 is also fixed to the graft 12.

In the case where the protrusion length is changed by moving the apex portion 30Aa of the movable ridge portion 30A in the axial direction relative to the graft 12, as described above, it becomes advantageous to increase the variable range of the protrusion length as the distance in the circumferential direction of the pair of fixed portions 40 continuous to the movable ridge portion 30A is increased. To increase the distance in the circumferential direction of the pair of fixed portions 40, for example, it is effective to increase the wavelength of the wave formed by the wave portion. In the present embodiment, the movable ridge portion 30A is provided on the second wave portion 34B which forms a wave shape of the second wavelength λ2 longer than the first wavelength λ1. Therefore, as compared with the case where the second wavelength λ2 is the same as the first wavelength λ1, it is advantageous to increase the variable range of the protrusion length of the movable ridge portion 30A (hooking portion 16).

In the first embodiment, an example the valley portion 36 which is provided between the fixed ridge portions 30B adjacent in the circumferential direction in one wave portion and is fixed to the graft 12 has been described. On the other hand, in the present embodiment, the valley portion 36 between the fixed ridge portions 30B adjacent in the circumferential direction in the first wave portion 34A (where the hook portion 52 is formed in the embodiment) is not connected to the graft 12 by being sutured, bonded, or the like, and its relative movement in the axial direction and the circumferential direction to the graft 12 is allowed. This advantage will be described.

The graft 12, when about to contract, moves so that the distance in the circumferential direction between the fixed ridge portions 30B adjacent in the circumferential direction is reduced. As a result, the valley portion 36 between the fixed ridge portions 30B is about to move relatively to the inner side in the axial direction relative to the graft 12. At this time, when the valley portion 36 is fixed to the graft 12, a resistance force F outwardly in the axial direction acts on the wave portion 34A from the fixed position, and it becomes difficult to contract the graft 12 due to the influence of the resistance force F. In this regard, when the valley portion 36 between the fixed ridge portions 30B is allowed to move relative to the graft 12 as in the present embodiment, the resistance force F may not be applied to the valley portion 36 when the graft 12 is about to contract. Thus, even when the fixed ridge portions 30B are included in the plurality of the ridge portions 30A and 30B, the graft 12 can be contracted by a light force. In addition, in the present embodiment, the same effect as the stent graft 10 of the first embodiment can be acquired.

Variations of the above components will be described.

The delivery devices 100 of the embodiments are only examples. Although the example of constraining the expansion of the distal end of the graft 12 with the end stent 14 by the stent constraining mechanism 108 has been described, the expansion of the proximal end of the graft 12 may be constrained with another end stent or a portion of the same stent. In addition, specific examples of each operation mechanism and the stent constraining mechanism 108 are not particularly limited. The plurality of stent receptacles 108a of the stent constraining mechanism 108 may be composed of the same member as in the embodiments, or a separate member.

In the embodiments, an example in which the conditions for the lower limit value of the maximum protrusion length L1 and the upper limit value of the maximum protrusion length L0 are both satisfied has been described. Not limited to this, only one of the conditions for L1 and L0 may or neither may be satisfied. The conditions for the shortening ratio may not be satisfied. In addition, the variable range of the protrusion length L of the graft 12 may be greater than zero to satisfy any of the conditions for the shortening ratio and the maximum protrusion lengths L1 and L0.

The specific configuration for changing the protrusion length L of the hooking portion 16 of the end stent 14 is not particularly limited.

The plurality of ridge portions 30A and 30B of the end stent 14 may not be configured by the wave portion 34. For example, the plurality of ridge portions 30A and 30B may be provided at the ends of a plurality of helical portions extending spirally in the axial direction.

The stent graft 10 may not include the connecting member 44. As the connecting member 44 that connects the intermediate line portion 30Ab of the movable ridge portion 30A to the graft 12, a cylindrical member secured to the graft 12 and allowing the intermediate line portion 30Ab to pass movably may be used.

The above embodiments and variations are exemplary. The technical concepts which abstract them should not be limited to the contents of the embodiments and variations. The contents of the embodiments and variations can be subject to many design changes, such as change, addition, or deletion of the components. In the aforementioned embodiments, the contents that can be subject to such design changes are emphasized by designating them as “embodiments”. However, design changes are allowed even without such designations. The hatching applied to the cross-section of the drawing does not limit the material of the hatched object. The structures and numbers referred to in the embodiments and variations naturally include those that can be considered the same in view of manufacturing errors.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A stent graft comprising:
a graft having a cylindrical shape; and
an end stent connected to at least an axial end of the graft, wherein
the end stent includes a hooking portion hooked on a stent constraining mechanism of a delivery device so as to constrain expansion of the end stent, and
the hooking portion is configured to shorten a protrusion length of a part protruding from the axial end to an outer side in an axial direction by expansion of the axial end together with the end stent.

2. The stent graft according to claim 1, wherein
when a state in which an outer diameter of the axial end is contracted to 50% of the outer diameter thereof in a case where the graft is in a maximum expansion state is called a reference contraction state, a maximum protrusion length L1 of the hooking portion when the graft is in the reference contraction state is no less than 1 mm.

3. The stent graft according to claim 1, wherein
a maximum protrusion length L0 of the hooking portion when the graft is in a maximum expansion state is no more than 5 mm.

4. The stent graft according to claim 1, wherein
when a state in which an outer diameter of the axial end is contracted to 50% of the outer diameter thereof in a case where the graft is in a maximum expansion state is called a reference contraction state, and a maximum protrusion length of the hooking portion when the graft is in the reference contraction state is called L1, and a maximum protrusion length of the hooking portion when the graft is in the maximum expansion state is called L0, a shortening ratio expressed by equation (1) below is no less than 50%:
shortening ratio (%)={(L1−L0)/L1}×100 (1).

5. The stent graft according to claim 1, wherein
a variable range of the protrusion length includes zero.

6. The stent graft according to claim 1, wherein
the end stent includes:
a movable ridge portion protruding to the outer side in the axial direction of the graft; and
a plurality of fixed portions provided continuously at the movable ridge portion on both sides in a circumferential direction relative to the movable ridge portion, the plurality of fixed portions being fixed to the graft, and
the movable ridge portion is movable relative to the graft at least at an apex portion in the axial direction, and constitutes the hooking portion.

7. The stent graft according to claim 6, wherein
the movable ridge portion includes an intermediate line portion provided between the apex portion and each of the plurality of fixed portions, the stent graft further comprising:
a connecting member configured to connect the intermediate line portion to the graft so as to be movable in a length direction of the intermediate line portion in relation to the graft.

8. The stent graft according to claim 6, wherein
the end stent includes a plurality of ridge portions each protruding to the outer side in the axial direction of the graft, the plurality of ridge portions being arranged in the circumferential direction of the graft, and
the plurality of ridge portions include the movable ridge portion and a fixed ridge portion fixed to the graft.

9. The stent graft according to claim 8, wherein
the movable ridge portion includes an intermediate line portion provided between the apex portion and each of the plurality of fixed portions, the stent graft further comprising:
a connecting member configured to connect the intermediate line portion to the graft so as to be movable in a length direction of the intermediate line portion in relation to the graft, and
the fixed ridge portion is connected to the graft on the outer side in the axial direction relative to a connection position of the movable ridge portion with the connecting member.

10. The stent graft according to claim 6, wherein
the end stent includes a plurality of ridge portions each protruding to the outer side in the axial direction of the graft, the plurality of ridge portions being arranged in the circumferential direction,
the plurality of ridge portions are formed by at least one wave portion extending in the circumferential direction and forming a wave shape,
the plurality of ridge portions include the movable ridge portion and a fixed ridge portion fixed to the graft, and
a valley portion between a plurality of the fixed ridge portions adjacent to each other in the circumferential direction in the wave portion is movable relative to the graft.

11. The stent graft according to claim 6, wherein
the end stent includes a plurality of ridge portions each protruding to the outer side in the axial direction of the graft, the plurality of ridge portions being arranged in the circumferential direction,
the plurality of ridge portions are formed by at least a plurality of wave portions each extending in the circumferential direction and forming a wave shape,
the plurality of wave portions include:
a first wave portion forming a wave shape of a first wavelength; and
a second wave portion forming a wave shape of a second wavelength longer than that of the first wavelength, and
the movable ridge portion is composed of a ridge portion of the second wave portion.
